(19) United States
(12) Patent Application Publication
(10) Pub. No.: US 2017/0007574 A1
(43) Pub. Date: Jan. 12, 2017

(54) PERIPHERAL KAPPA OPIOID RECEPTOR AGONISTS FOR UREMIC PRURITUS IN DIALYSIS PATIENTS

(71) Applicant: Cara Therapeutics, Inc., Stamford, CT (US)

(72) Inventors: Robert H. SPENCER, New Hope, PA (US); Frédérique MENZAGHI, Rye, NY (US); Derek T. CHALMERS, Riverside, CT (US)

(73) Assignee: Cara Therapeutics, Inc., Stamford, CT (US)

(21) Appl. No.: 15/193,837

(22) Filed: Jun. 27, 2016

Related U.S. Application Data
(60) Provisional application No. 62/188,189, filed on Jul. 2, 2015.
(60) Provisional application No. 62/195,857, filed on Jul. 23, 2015.

(51) Int. Cl. A61K 31/40; A61K 9/00

(52) U.S. Cl. CPC A61K 31/40; A61K 9/0019

(57) ABSTRACT

The invention provides a method of prevention, inhibition or treatment of uremic pruritus in a dialysis patient by administering an effective amount of a kappa opioid receptor agonist. Also provided is a method of inhibition or treatment of adverse symptoms associated with dialysis affecting the quality of life of dialysis patient, the method includes administering an effective amount of a kappa opioid receptor agonist. The adverse symptoms associated with dialysis addressable by the methods of the invention include uremic pruritus, sleep disruption, depression and other mood alterations.

Pruritus, or itching, occurs in many diseases and conditions such as, for instance, ocular pruritus associated with conjunctivitis, as well as pruritus associated with dermatological conditions such as eczema (dermatitis), including atopic or contact dermatitis, psoriasis, polycythemia vera, lichen planus, lichen simplex chronicus, pediculosis (lice), thyrotoxicosis, tinea pedis, urticaria, scabies, vaginitis, anal pruritus associated with hemorrhoids, as well as insect-bite pruritus and drug-induced pruritus, such as pruritus induced by mu opioids, including morphine. Pruritus is also associated with chronic kidney dysfunction, including end-stage renal disease, where many patients are receiving kidney dialysis, and other forms of cholestasis, including primary biliary cirrhosis, intrahepatic cholestasis of pregnancy, chronic cholestatic liver disease, uremia, malignant cholestasis, and jaundice.

Uremic pruritus, also called chronic kidney disease-associated pruritus, is common in patients suffering from chronic kidney dysfunction, occurring in about 20%-50% of patients with renal failure. Uremic pruritus is a chronic itching condition causing long-term pain and suffering, especially in patients with advanced or end-stage renal disease. Currently, this condition is managed by optimizing regimens to provide adequate dialysis of the patient's blood. Topical emollients are used in patients with localized itching and antihistamines delivered orally have been found to provide some, though limited benefits in dialysis patients. Currently, the only cure is a kidney transplant which is not available to most patients due to limited organ availability, tissue matching requirements and the high costs of surgery and post-surgical therapy.

### SUMMARY

The present invention provides a method of treatment of a patient suffering from uremic pruritus including administering an effective amount of a kappa opioid receptor agonist to a patient undergoing a haemodialysis regimen on at least one of the days in which the dialysis procedure occurs. The invention also provides a method of reducing an adverse symptom associated with dialysis in a patient undergoing a haemodialysis regimen, including administering an effective amount of a kappa opioid receptor agonist.

### BRIEF DESCRIPTION OF THE FIGURES

FIG. 1 is a schematic of the study design of Part A of the clinical trial of CR845 in patients with uremic pruritus.

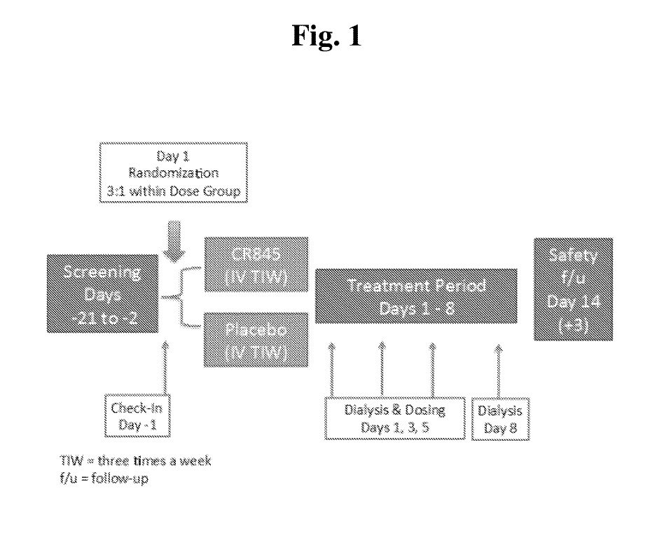

FIG. 2 is a schematic of the study design of Part B of the clinical trial of CR845 in patients with uremic pruritus.

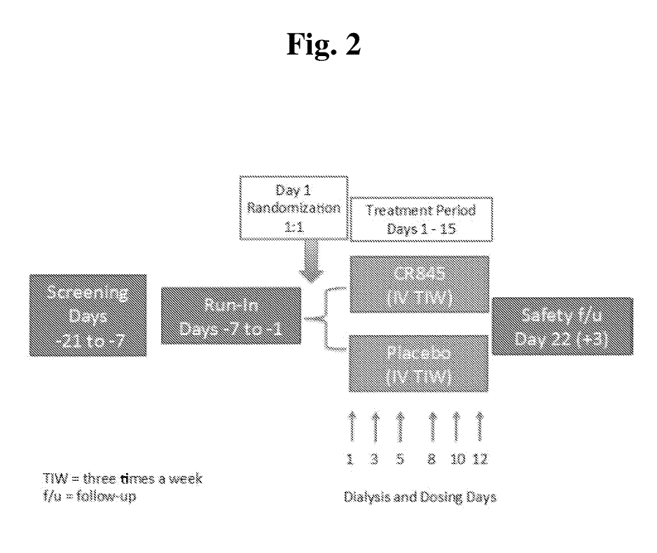

FIG. 3 shows the pharmacokinetics of CR845 in nineteen dialysis patients suffering from end stage renal disease (ESRD), exhibiting a half-life of ˜24 hrs. CR845 levels in dialysis patient blood is plotted at 5, 15 and 30 minutes, 1, 2, 4 6, 8 12 and 24 hours after post-dialysis administration of CR845. CR845 levels at day 3 and at day 5 pre-dialysis and post-dialysis before administration of CR845 are interpolated between days 1 and 5.

FIG. 4 is a histogram comparing change in itch from baseline on a visual analog scale (VAS) in placebo-treated and CR845-treated dialysis patients with end stage renal disease (ESRD). Bars represent standard error about the mean (S.E.M.).

FIG. 5 is a histogram comparing change in itch in placebo-treated and CR845-treated dialysis patients with end stage renal disease (ESRD) during the run-in period, at week 1 and week 2 of the CR845 treatment trial. Bars represent standard error about the mean (S.E.M.).

FIG. 6 shows the progress of the reduction in “worst itching” average over daytime and night time as assessed by a visual analog scale (VAS) in CR845-treated dialysis patients over the 15 days of the trial. Bars represent standard error about the mean (S.E.M.).

FIGS. 7A and 7B show the significant reduction in Worst itch intensity reported for day time and night time, respectively in CR845-treated patients by the second week of the trial. The reductions in levels of itch in CR845-treated patients assessed on a visual analog scale (VAS) is significant (p<0.05) in the daytime and highly significant (p<0.01) in the night time by the second week of the trial. Bars represent standard error about the mean (S.E.M.).

FIG. 8 is a histogram comparing change in itch-related quality of life assessed as the Skindex-10 score in placebo-treated and CR845-treated dialysis patients. Bars represent standard error about the mean (S.E.M.).

FIG. 9 shows the improvement in Skindex-10 (reduction is an improvement on this scale) in itch-related quality of life scores. in placebo and CR845-treated dialysis patients. Bars represent standard error about the mean (S.E.M.).

FIG. 10 shows the reduction in itch-related sleep disturbances assessed on the itch MOS Sleep problems Index II (SLP-9). Bars represent standard error about the mean (S.E.M.).

### DETAILED DESCRIPTION

The invention provides a method of prevention, inhibition or treatment of a patient suffering from uremic pruritus: the method includes administering an effective amount of a kappa opioid receptor agonist to a patient undergoing a dialysis regimen on one or more days in which the dialysis occurs. The weekly schedule for administration of the kappa opioid receptor agonist may be on one dialysis day, or two or three of the dialysis days. In one embodiment of the kappa opioid receptor agonist regimen, the kappa opioid receptor agonist is administered three times per week for at least one week.

In another embodiment of the kappa opioid receptor agonist treatment regimen, at least one administration of the kappa opioid receptor agonist is administered within one hour after a dialysis treatment. In another embodiment at least one administration of the kappa opioid receptor agonist is within fifteen minutes after a dialysis procedure. In one embodiment the kappa opioid receptor agonist may be administered by intravenous injection, e.g. in an IV bolus injection.

In another embodiment the effective amount of the kappa opioid receptor agonist can be estimated from a patient's dry weight. For instance, the effective amount of the kappa opioid receptor agonist administered may be from about 0.1 μg/kg of patient's dry weight to about 5.0 μg/kg of patient's dry weight. In another embodiment, the effective amount of the kappa opioid receptor agonist administered may be from about 0.5 μg/kg of patient's dry weight to about 2.5 μg/kg of patient's dry weight. In another embodiment, the effective amount of the kappa opioid receptor agonist administered is about 1.0 μg/kg of patient's dry weight. In another embodiment, the effective amount of the kappa opioid receptor agonist administered is about 2.5 μg/kg of patient's dry weight.

The present invention also provides a method of reducing an adverse symptom associated with dialysis in a patient undergoing dialysis, the method includes administering an effective amount of a kappa opioid receptor agonist to the patient. Adverse symptoms that may be prevented, inhibited or treated by the methods of the present invention include uremic pruritus, sleep disruption, and mood alteration. The sleep disruption may be pruritus-associated sleep disruption, wherein the patient is roused from sleep by itching. In one embodiment, the mood alteration that may be inhibited or treated by the methods of the present invention is depression.

In one embodiment, the invention further provides a method of prevention, inhibition or treatment of a patient suffering from uremic pruritus: the method includes administering an effective amount of a peripherally-restricted kappa opioid receptor agonist to a patient undergoing a dialysis regimen on one or more days in which the dialysis occurs.

In another embodiment the invention provides a method of prevention, inhibition or treatment of a patient suffering from uremic pruritus: the method includes administering an effective amount of a long acting kappa opioid receptor agonist to a patient undergoing a dialysis regimen on one or more days in which the dialysis occurs; the long acting kappa opioid receptor agonist can be any long acting kappa opioid receptor agonist, such as for instance, and without limitation, a synthetic peptide amide of formula I:

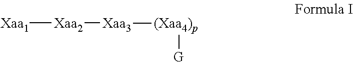

wherein

(i) G is

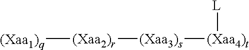

(ii) G is

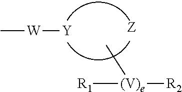

or

(iii) G is

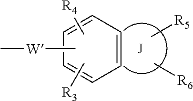

In another embodiment, the invention provides a method of prevention, inhibition or treatment of a patient suffering from uremic pruritus: the method includes administering an effective amount of CR845 to a patient undergoing a dialysis regimen on one or more days in which the dialysis occurs.

### DEFINITIONS

The term: kappa opioid receptor—as used herein means the class of opioid receptors designated “Kappa” or “κ” of the four classes of receptors (Mu, Kappa, Delta and ORL1, a receptor type identified by genetic homology) that bind opiates, found in the brain, spinal column and peripheral neurons.
The term: kappa opioid receptor agonist—means a compound that binds and activates a kappa opioid receptor. CR845 is an example of a kappa opioid receptor agonist. Other kappa opioid receptor agonists include asimadoline, TRK-820, Remitch® and nalbuphine. Asimadoline, a selective, non-peptidic kappa-opioid receptor agonists is also useful in methods according to the present invention Asimadoline has the diarylacetamide structure shown below:

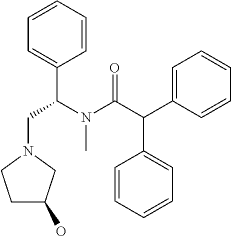 [SMILES: CN(C(=O)C(c1ccccc1)c1ccccc1)[C@H](CN1CC[C@H](O)C1)c1ccccc1]

(N-[(1S)-2-[(3S)-3-hydroxypyrrolidin-1-yl]-1-phenylethyl]-N-methyl-2,2-diphenylacetamide).
Nalfurafine (also known as AC-820, TRK-820) is a kappa opioid receptor agonist marketed for orally delivered opioid treatment for uremic pruritus in hemodialysis patients. Nalfurafine is another kappa opioid receptor agonist useful according to the present invention. Nalfurafine is (2E)-N-[(5α,6β)-17-(cyclopropylmethyl)-3,14-dihydroxy-4,5-epoxymorphinan-6-yl]-3-(3-furyl)-N-methylacrylamide, and has the following chemical structure:

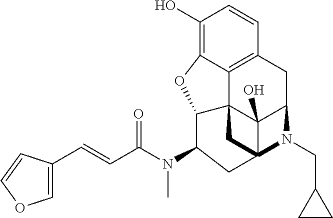

The term: peripheral kappa opioid receptor agonist—means a peripherally-restricted compound (e.g. CR845 or other member of the class of D-amino acid peptide amides disclosed in U.S. Pat. Nos. 7,402,564; 7,727,963; 8,217,007; 8,951,970; and 7,713,937) that binds and activates a kappa opioid receptor, i.e. a kappa opioid receptor agonist that shows little or no CNS effects.
The term: effective amount—means an amount that is sufficient to provide the stated effect.
The term: patient's dry weight—means the weight of the patient when in the normal hydration state, i.e. not suffering from excess fluid retention. The patient's dry weight is usually estimated by a physician and is intended to be similar to the patient's weight with normal kidney function after urinating. It is the lowest weight that the patient can safely reach after dialysis without developing symptoms of low blood pressure, such as cramping, which can occur when too much fluid is removed.
The term: dose means the amount by weight of therapeutic compound (e.g. CR845) administered.
The term: (μg/kg) dry weight—means the amount in micrograms per kilogram of patient's dry weight as defined above.
The term: dialysis interchangeably referred to herein as haemodialysis—means the artificial filtration process commonly provided for patients suffering from renal dysfunction.
The term: regimen/schedule—means the dose and timing of administration to the patient of a therapeutic compound.
The term: I.V. bolus injection—means injection of discrete amount to achieve an effective concentration, e.g. by I.V. push.
The term: adverse symptom—means an unfavorable departure from normal signs, e.g. nausea, dizziness, dry mouth, chills and shivering, etc.

### Kappa Opioid Receptor Agonists

One class of kappa opioid receptor agonists is represented by the synthetic D-peptide amides and dimers thereof, and conjugates that have been described (See U.S. Pat. No. 7,402,564). Clinical compound, CR845 is a member of this class of kappa opioid receptor agonists.

The synthetic peptide amides and dimers thereof have the structure of formula I:

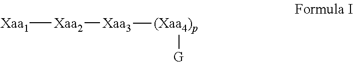

In formula I:
each Xaa₁ is independently chosen from the following D-amino acids: (A)(A′)D-phenylalanine, (A)(A′)α-methyl-D-phenylalanine, D-tyrosine, D-1,2,3,4-tetra-hydroisoquinoline-3carboxylic acid, D-tert-leucine, D-neopentylglycine, D-phenylglycine, D-homo-phenylalanine, and β-(E)D-alanine, wherein each (A) and each (A′) are phenyl ring substituents independently chosen from —H, —F, —Cl, —NO₂, —CH₃, —CF₃, —CN, —CONH₂, and wherein each (E) is independently chosen from cyclobutyl, cyclopentyl, cyclohexyl, pyridyl, thienyl and thiazolyl;
each Xaa₂ is independently chosen from (A)(A′)D phenylalanine, 3,4-dichloro-D-phenylalanine, (A)(A′)(α-Me)D-phenylalanine, D-1-naphthylalanine, D-2-naphthylalanine, D-tyrosine, (E)D-alanine, and D-tryptophan;
each Xaa₃ is independently chosen from D-norleucine, D-phenylalanine, (E)-D-alanine, D-leucine, α-MeD-leucine, D-homoleucine, D-valine, and D-methionine;
each Xaa₄ is independently chosen from (B)2D-arginine, (B)2D-norarginine, (B)2D-homoarginine, ζ-(B)D-homolysine, D-2,3-diaminopropionic acid, ε-(B)D-lysine, ε-(B)2-D-lysine, D-aminomethylphenyl-alanine, amidino-D-aminomethyl-phenylalanine, γ-(B)2D-α,γ-diaminobutyric acid, δ-(B)2α-(B′)D-ornithine, D-2-amino-3(4-piperidyl)-propionic acid, D-2-amino-3(2-aminopyrrolidyl)propionic acid, D-α-amino-β-amidino-propionic acid, α-amino-4-piperidineacetic acid, cis-α,4-diaminocyclo-hexane acetic acid, trans-α,4-diaminocyclohexaneacetic acid, cis-α-amino-4-methyl-aminocyclo-hexane acetic acid, trans-α-amino-4-methylaminocyclohexane acetic acid, α-amino-1-amidino-4-piperidineacetic acid, cis-α-amino-4-guanidino-cyclohexane acetic acid, and trans-α-amino-4-guanidinocyclohexane acetic acid, wherein each (B) is independently chosen from —H and C₁-C₄ alkyl, and (B′) is —H or (α-Me);
and p is zero or 1; such that when p is 1 G is bonded to Xaa₄ and when p is zero, then G is directly bonded to Xaa₃.

The moiety G of formula I is selected from one of the following moieties (i)-(iiii):

### (i) G is

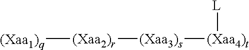

wherein p, q, r, s and t are each independently zero or 1, provided that at least one of s and t is 1, such that when t is 1 L is bonded to Xaa₄ and when t is zero, then L is directly bonded to Xaa₃. The moiety L is a linker chosen from ε-D-Lys, ε-Lys, δ-D-Orn, δ-Orn, γ-aminobutyric acid, 8-aminooctanoic acid, 11-amino-undecanoic acid, 8-amino-3,6-dioxaoctanoic acid, 4-amino-4-carboxylic piperidine and bis(D-Lys-Gly)Lactam.

### (ii) G is

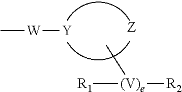

and p is 1.

The linking moiety, W can be any of the following three alternatives: (a) null, provided that when W is null, Y is nitrogen and is bonded to the C-terminus of Xaa₄ to form an amide; (b) —NH—(CH₂)_{b}— with b equal to 0, 1, 2, 3, 4, 5, or 6; or (c) —NH—(CH₂)_{c}—O— with c equal to 2, or 3, provided that Y is carbon.

In each of the foregoing alternatives, (b) and (c) the nitrogen atom of W is bonded to the C-terminus of Xaa₄ to form an amide; and the moiety

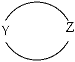

is an optionally substituted 4-, 5-, 6-, 7-, or 8-membered heterocyclic ring moiety wherein Y is a carbon or a nitrogen atom and Z is carbon, nitrogen, oxygen, sulfur, sulfoxide, or sulfonyl; provided that when such ring moiety is a six, seven or eight-membered ring, Y and Z are separated by at least two ring atoms, and provided further that when such ring moiety is aromatic, then Y is a carbon atom.

The moiety V in the substituent of the Y—Z-containing ring in formula I is a C₁-C₆ alkyl linker when present. The operator, e is zero or 1, such that when e is zero, then V is null, and R₁ and R₂ are directly bonded to the same or different ring atoms.

The moiety V represents C₁-C₆ alkyl, and the operator, e is either zero or 1, wherein when e is zero, then V is null and, R₁ and R₂ are directly bonded to the same or different ring atoms. The groups R₁ and R₂ can be any one of (a), (b), (c) or (d) as follows:
(a) R₁ is H, OH, halo, CF₃, —NH₂, —COOH, C₁-C₆ alkyl, C₁-C₆ alkoxy, amidino, C₁-C₆ alkyl-substituted amidino, aryl, optionally substituted heterocyclyl, Pro-amide, Pro, Gly, Ala, Val, Leu, Ile, Lys, Arg, Orn, Ser, Thr, CN, CONH₂, COR′, SO₂R′, CONR′R″, NHCOR′, OR′, or SO₂NR′R″;
wherein said optionally substituted heterocyclyl is optionally singly or doubly substituted with substituents independently selected from the group consisting of C₁-C₆ alkyl, —C₁-C₆ alkoxy, oxo, —OH, —Cl, —F, —NH₂, —NO₂, —CN, —COOH, and amidino;
wherein R′ and R″ are each independently H, C₁-C₈ alkyl, aryl, heterocyclyl or R′ and R″ are combined to form a 4-, 5-, 6-, 7-, or 8-membered ring, which ring is optionally substituted singly or doubly with substituents independently selected from the group consisting of C₁-C₆ alkyl, —C₁-C₆ alkoxy, —OH, —Cl, —F, —NH₂, —NO₂, —CN, and —COOH, amidino; and R₂ is H, amidino, singly or doubly C₁-C₆ alkyl-substituted amidino, —CN, —CONH₂, —CONR′R″, —NHCOR′, —SO₂NR′R″, or —COOH;
(b) R₁ and R₂ taken together can form an optionally substituted 4-, 5-, 6-, 7-, 8- or 9-membered heterocyclic monocyclic or bicyclic ring moiety which is bonded to a single ring atom of the Y and Z-containing ring moiety;
(c) R₁ and R₂ taken together with a single ring atom of the Y and Z-containing ring moiety can form an optionally substituted 4-, 5-, 6-, 7-, or 8-membered heterocyclic ring moiety to form a spiro structure; or
(d) R₁ and R₂ taken together with two or more adjacent ring atoms of the Y and Z-containing ring moiety can form an optionally substituted 4-, 5-, 6-, 7-, 8- or 9-membered hetero-cyclic monocyclic or bicyclic ring moiety fused to the Y and Z-containing ring moiety.

Each of the aforementioned optionally substituted 4-, 5-, 6-, 7-, 8- or 9-membered heterocyclic ring moieties that include R₁ and R₂ is optionally singly or doubly substituted with substituents independently chosen from C₁-C₆ alkyl, C₁-C₆ alkoxy, optionally substituted phenyl (as defined above), oxo, —OH, —Cl, —F, —NH₂, —NO₂, —CN, —COOH, and amidino.

In the first of four alternative embodiments, the moiety R₁ in formula I can be any of the following groups: —H, —OH, halo, —CF₃, —NH₂, —COOH, C₁-C₆ alkyl, C₁-C₆ alkoxy, amidino, C₁-C₆ alkyl-substituted amidino, aryl, optionally substituted heterocyclyl, Pro-amide, Pro, Gly, Ala, Val, Leu, Ile, Lys, Arg, Orn, Ser, Thr, CN, CONH₂, COR′, SO₂R′, CONR′R″, NHCOR′, OR′, or SO₂NR′R″;
wherein the optionally substituted heterocyclyl is optionally singly or doubly substituted with substituents independently chosen from C₁-C₆ alkyl, C₁-C₆ alkoxy, oxo, —OH, —Cl, —F, —NH₂, —NO₂, —CN, —COOH, and amidino.

The moieties R′ and R″ are each independently H, C₁-C₈ alkyl, aryl, or heterocyclyl. Alternatively, R′ and R″ can be combined to form a 4-, 5-, 6-, 7-, or 8-membered ring, which ring is optionally substituted singly or doubly with substituents independently chosen from C₁-C₆ alkyl, C₁-C₆ alkoxy, —OH, —Cl, —F, —NH₂, —NO₂, —CN, —COOH and amidino. The moiety R₂ can be any of —H, amidino, singly or doubly C₁-C₆ alkyl-substituted amidino, —CN, —CONH₂, —CONR′R″, —NHCOR′, —SO₂NR′R″, or —COOH.

In a second alternative embodiment, the moieties R₁ and R₂ taken together can form an optionally substituted 4-, 5-, 6-, 7-, 8- or 9-membered heterocyclic monocyclic or bicyclic ring moiety which is bonded to a single ring atom of the Y and Z-containing ring moiety.

In a third alternative embodiment, the moieties R₁ and R₂ taken together with a single ring atom of the Y and Z-containing ring moiety can form an optionally substituted 4-, 5-, 6-, 7- or 8-membered heterocyclic ring moiety to form a spiro structure.

In a fourth alternative embodiment, the moieties R₁ and R₂ taken together with two or more adjacent ring atoms of the Y and Z-containing ring moiety can form an optionally substituted 4-, 5-, 6-, 7-, 8- or 9-membered heterocyclic monocyclic or bicyclic ring moiety fused to the Y and Z-containing ring moiety.

In formula I in the above second, third and fourth alternative embodiments, each of the optionally substituted 4-, 5-, 6-, 7-, 8- and 9-membered heterocyclic ring moieties comprising R₁ and R₂ can be singly or doubly substituted with substituents independently chosen from C₁-C₆ alkyl, C₁-C₆ alkoxy, optionally substituted phenyl, oxo, —OH, —Cl, —F, —NH₂, —NO₂, —CN, —COOH and amidino.
(iii) G is

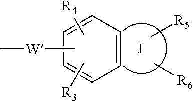

wherein J is a 5-, 6-, or 7-membered heterocyclic ring moiety comprising 1, 2, or 3 heteroatoms in the ring wherein R₃ and R₄ are each independently selected from H, C₁-C₃ alkyl, halo, —OH, —CF₃, —NH₂, —COOH and amidino; and R₅ and R₆ are each independently selected from H, C₁-C₃ alkyl, oxo, halo, —OH, —CF₃, —NH₂, —COOH and amidino.

The moiety W′ is chosen from the following two options: —NH—(CH₂)_{b}— with b equal to zero, 1, 2, 3, 4, 5, or 6; and —NH—(CH₂)_{c}—O— with c equal to 2 or 3.

The above formula I definitions are subject to the following three provisos:
(1) That when the Y and Z-containing ring moiety is a six or seven membered ring having a single ring heteroatom and such heteroatom is N, and e is zero, then R₁ is not OH, and R₁ and R₂ are not both H;
(2) That when the Y and Z-containing ring moiety is a six membered ring comprising two ring heteroatoms, both Y and Z are nitrogen atoms, W is null, and the moiety —Vₑ(R₁)(R₂) is attached to Z, then —Vₑ(R₁)(R₂) is chosen from amidino, C₁-C₆ alkyl-substituted amidino, dihydroimidazole, —CH₂COOH, and —CH₂C(O)NH₂; and
(3) That if the Y and Z-containing ring moiety is a six membered ring comprising a sulfur or an oxygen ring heteroatom, or if the Y and Z-containing ring moiety is a non-aromatic six membered ring that includes two ring heteroatoms, wherein both Y and Z are nitrogen atoms and W is null, or if the Y and Z-containing ring moiety is a six-membered aromatic ring that includes a single ring heteroatom, which heteroatom is a nitrogen atom, then, when e is zero, R₁ and R₂ are not both hydrogen.

One example of the kappa opioid receptor agonist synthetic peptide amides is CR845:

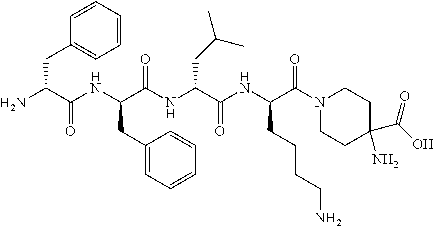 [SMILES: CC(C)C[C@@H](NC(=O)[C@@H](Cc1ccccc1)NC(=O)[C@H](N)Cc1ccccc1)C(=O)N[C@H](CCCCN)C(=O)N1CCC(N)(C(=O)O)CC1]

CR845: D-Phe-D-Phe-D-Leu-D-Lys-[ω(4-aminopiperidine-4-carboxylic acid)]-OH

Asimadoline is a kappa opioid receptor agonist that acts that has been investigated as a possible treatment for irritable bowel syndrome (IBS). See Szarka et al., Clin. Gastroenterol. Hepatol. 200S7 November; 5(11):1268-1275.

Nalbuphine—(Nubain®) is a dual mu and kappa opioid receptor agonist that has been tested in an extended release formulation in hemodialysis patients as a potential therapy for pruritus. See Hawi et al. (2015) BMC Nephrology 16:47.

Remitch® (Nalfurafine HCl) from Toray Industries is a kappa opioid receptor agonist that has also been investigated in an oral formulation as a possible treatment for pruritus in hemodialysis patients. See Kumagai et al. Am. J. Nephrol. 2012; 36(2):175-183.

Surprisingly, the method of prevention, inhibition or treatment of a patient suffering from uremic pruritus of the invention, that includes administering an effective amount of CR845 to a patient undergoing a dialysis regimen on one or more days in which the dialysis occurs, results in an unexpectedly sustained, long lasting reduction in pruritus in the patient.

Without wishing to be bound by theory, it is believed that this sustained and long lasting reduction in pruritus is due to the unique pharmacokinetics of CR845 and related D-amino acid peptide amides, which are processed by the kidney and excreted in the urine in normal individuals. Thus, dialysis patients suffering kidney dysfunction do not excrete CR845, which remains in the blood and is only cleared by the dialysis procedure. After administration of an effective amount of CR845 within 15 minutes, 30 minutes or up to one to two hours after dialysis, patients experience surprisingly low levels of pruritus.

Remarkably, the reduction in pruritus as judged by the patient on a visual analog scale (VAS), can be greater than 50%, in many cases greater than 75%, in a significant number of cases greater than 90%, and occasionally reach as much as a 98% reduction in patient assessed itch on the visual analog scale. Before the present invention, such high levels of relief from itching were not achievable.

Furthermore, administration of CR845 provides relief from other dialysis-associated adverse symptoms in addition to pruritus, such as but not limited to sleep disorders, including sleep disruption, moodiness and depression.

### Examples

A multi-center, randomized, double-blind, placebo-controlled, study (CR845-CLIN2005) was conducted in two parts to evaluate the safety, Pharmacokinetics (PK), and efficacy of repeated doses of CR845 administered as intravenous (I.V.) boluses to haemodialysis patients.

Part A was conducted in a clinical research unit with capabilities of performing haemodialysis and keeping patients overnight. Patients in Part A received one of three doses of CR845 or placebo in a dose escalation following a sequential group design.

Part B was conducted in outpatient dialysis units where patients were normally dialyzed. Patients in Part B received CR845 or placebo in a parallel group design.

### Study Design Part A

One of three doses of CR845 or matched placebo were administered as an IV bolus once immediately after each dialysis session for one week. Each dose cohort was comprised of twenty-four patients (6 CR845 and 2 placebo for each of three dose levels: See below). Part A consisted of a Screening visit, Treatment period, and Follow-up visit (approximately 1 week after the last dose). Vitals signs, physical examinations, 12-lead ECG, clinical laboratory tests and urine output in patients who were not anuric (i.e., at least 1 cup/day of urine output by history) were monitored periodically and adverse events (AEs) and concomitant medications were continuously recorded during the study.

### Study Design Part B

One dose of CR845 or placebo was administered once immediately after each dialysis session for a period of 2 weeks in 65 patients (blindly randomized to approximately 50% of the patients in the CR845 group and approximately 50% patients in the placebo group).

Part B consists of a Screening visit, a one-week Run-in Period (Baseline), a Treatment Period of 2 weeks and a Follow-up Visit (approximately 1 week after the last dose). Patients report Daytime and Nighttime Worst Itching VAS scores daily during the entire Treatment Period. In addition, during selected study visits, patients completed their additional patient reported outcomes (PROs) (i.e., Itch MOS, Patient Self-Categorization of Pruritus Disease Severity and Skindex-10). Vital signs, physical examinations, 12-lead ECG, and clinical laboratory tests were monitored periodically and AEs and concomitant medications were recorded continuously during the study.

### Endpoints

### Safety Endpoint (Parts A and B)

The safety endpoint is the overall safety and tolerability of CR845 as assessed by the frequency and severity of AEs by treatment group, physical examination, vital signs, 12-lead ECG, and clinical safety laboratory evaluations. All patients receiving any study drug were included in the safety analysis.

Pharmacokinetic Endpoints (Part A): The PK profile of CR845 with dosing after each dialysis session over a 1 week treatment period (three times per week) was recorded.

Primary Efficacy Endpoints (Part B): The change from baseline to the average of Week 2 worst itching (daytime and night time) visual analog scale (VAS). The mean of the last 8 VAS scores from Week 2 was used for comparison.

Secondary Efficacy Endpoints (Part B): The change from baseline to Day 15 in itch-related quality of life as assessed by the total Skindex-10 scores; Change from baseline to Day 15 in itch-related sleep disturbance based on the Itch MOS sleep problems index II (SLP-9); and Percentage of patients with Patient Assessed Self-categorization of Pruritus Disease Severity with a reduction in category from “B” or “C” from Screening to Day 15.

### Duration of Treatment

In Part A, the duration of treatment for each individual patient was one week for a total of three doses of study drug. The overall study duration for each individual patient in Part A was about 5.5 weeks.

In Part B, the duration of treatment for each individual patient was two weeks for a total of six doses of study drug. The overall study duration for each individual patient in Part B was up to 6.5 weeks.

### Study Procedures

### Part A

### Screening

To be included in the trial, the patient had to have completed screening within 21 days prior to the beginning of the Treatment Period. After written informed consent was obtained, the following procedures and assessments were run:

Medical and medication history were recorded: Any adverse events (AEs) that occurred during the Screening Period following signing of the informed consent form (ICF) were recorded as Medical History. Height, weight, vital signs, including BP, HR, RR, and temperature and physical examination results, including examination of the heart, lungs, abdomen, extremities, neurological system, and vascular system were recorded. A blood sample was taken for central laboratory pregnancy test for women of child-bearing potential or FSH assay for post menopausal women and a drug test was performed. Patient Questionnaires: VAS Worst Itching Day and Night for prior 24 hour period was completed in the clinic, within 6 hours prior to starting dialysis whenever possible.

### Treatment Period

Part A—Day −1 Admission to clinic: Patients were admitted 24 hours prior to the Day 1 dialysis. This day, Day −1 is the day prior to the patient's usual dialysis day. The 24-hour urine collection was begun and fluid intake was recorded. The urine collection was planned so that the collection was completed prior to starting dialysis on the following day. The total 24-hour urine volume was recorded and an aliquot submitted to the laboratory for urine sodium and creatinine measurement. Fluid intake (oral and I.V.) was recorded during the same 24-hour period as urine volume. Patient questionnaires: VAS Worst Itching Day and Night for prior 24 hour period were completed in the clinic. The erythropoiesis stimulating agent was administered to the patient after fasting overnight in the clinical research unit. Concomitant medications were recorded.

### Dialysis

The dialysis prescription was kept constant throughout the study, unless absolutely necessary for patient safety. All procedural data (start and stop times, net ultrafiltration, access changes, dialysis bath sodium concentration, ESA usage) were recorded.

### Part A

Study drug was administered as an I.V. bolus via I.V. push into dialysis venous line (e.g., into the venous port) within 15 minutes following the end of dialysis (i.e., return of blood to the patient) on scheduled drug administration days. Following the bolus, the venous line was flushed with at least 10 mL of normal saline. The patient's estimated dry weight (i.e., the target post-dialysis weight, as determined by the patient's nephrologist or dialysis unit) was used to calculate the dose of the study drug.

Individual doses of CR845 or placebo were prepared by an unblinded pharmacist (or qualified staff) from one vial in a sterile environment (e.g. a sterile hood) by withdrawal of a patient-specific volume of CR845 or placebo, up to 24 hours prior to administration. Doses for patients in Group 1 (0.5 μg/kg) were prepared from CR845 0.05 mg/mL; doses for patients in Group 2 (1 μg/kg) were prepared from CR845 0.10 mg/mL; and doses for patients in Group 3 (2.5 μg/kg) were prepared from CR845 0.25 mg/mL.

Placebo doses for each group were prepared from the placebo vials. For each dose, an appropriate volume of CR845 or placebo solution (based upon the patient's estimated dry weight) was drawn up from 1 vial using a sterile syringe (1 mL or 3 mL Plastipak polypropylene syringe, Becton Dickinson) and a 21 G×1.5 inch sterile needle (Becton Dickinson). The needle was removed from the filled syringe and the filled syringes were capped (Braun Combi-stoppers, polyethylene) and the final solution for administration was stored for up to 24 hours at 2 to 8° C.

As each dose was proportional to the concentration of CR845 in the vial (including placebo), the volume of study drug administered was dependent only upon the patient's estimated dry weight. Prepared syringes were blinded, and thus, the Investigator and study staff remained blinded to the treatment groups.

### Part B (Days 3, 5, 10 and 12)

For Part B, the dose of 2.5 μg/kg (to be confirmed upon completion of Part A) or placebo was administered as a single I.V. bolus three times a week post-dialysis for two weeks.

### Selection of Doses Used in the Study

The combined safety and PK data from an ascending Phase 1 dose ranging study of I.V. doses of 0.001 to 0.006 mg/kg in haemodialysis patients (Study CR845-CLIN1003) provided the basis for the selection of the doses of CR845 used in this study.

### Prior and Concomitant Medications

Prior medications were defined as those that the patient has taken during the 15 days prior to the Screening Visit through prior to the first dose of study drug on Day 1. Concomitant medications were medications that are taken from after the start of the first dose of study drug on Day 1 through the end of the study (i.e. follow up visit).

All prior and concomitant medications, including over-the-counter (OTC) medications used by patients during this study, were recorded in the appropriate source documents at each study visit and recorded on the appropriate page of the case report form (CRF).

Patients taking gabapentin, calcineurin inhibitors, opioids; antipsychotics; systemic or topical corticosteroids (other than otic or ophthalmic preparations); sedatives; hypnotics; antianxiety agents; SSRIs; or tricyclic antidepressants were required to remain on the same drugs at the same doses through the end of Week 2 unless a significant change in the patient's medical status necessitates a change.

### Efficacy Assessment (Part B Only)

The effect of CR845 on itch was measured by means of the following PROs:
Worst Itching on Visual Analog Scale (VAS);
Patient's Self-categorization of Pruritus Disease Severity;
Itch Medical Outcomes Study (MOS) sleep questionnaire;
Skindex-10;
Patient's treatment satisfaction.

### Visual Analog Scale

Intensity of itch was measured using a 100-mm visual analog scale (VAS), in which the patient was asked to mark a line that represents the severity of their itch for the assessment time point, where “0=no itch” and “100=worst itch you can imagine”. Patients recorded their itch assessment scores on a worksheet under the direction of the site study staff, as defined below. The VAS has been widely utilized for evaluation of pruritus, including, uremic pruritus (See Refs^{3,4,5,6}).

### Self-Categorization of Pruritus Disease Severity

Patients were asked to complete the Self-categorization of a Pruritus Disease Severity (PDS) questionnaire. Patients select a patient profile (A, B or C) that most closely resembles their profile, with profile A being the least affected by itch and profile C being the most affected by itch. Patients who classified themselves as Patient B or Patient C are eligible to qualify for inclusion in the study. This system of self-categorization of PDS was tested previously in a longitudinal study of uremic pruritus and found to correlate with both itch intensity and measurements from instruments evaluating quality of life (Ref.4).

### Itch MOS Sleep Questionnaire

An Itch MOS sleep questionnaire was adapted from the Medical Outcomes Study (MOS) sleep survey in order to measure sleep disturbance as a result of nocturnal itching (Ref.4). For most questions, patients circled one of six numbers ranging from “1” (“all of the time”) to “6” (“none of the time”), indicating the frequency of various aspects of pruritus-related sleep disruption over the preceding week. Patients also estimated the average amount of sleep per night during the past week. The SLP-9 scoring method was utilized. The Itch MOS sleep questionnaire has been tested previously in a longitudinal study of uremic pruritus and found to correlate with both itch intensity as well as evaluating quality of life generally and to other measures of sleep and mood (Ref.4).

### Skindex-10

Skindex-10 is a validated questionnaire developed specifically for uremic pruritus, for measurement of quality of life (Ref.4). Patients were asked to fill in one of seven bubbles (“0 [never bothered], 1, 2, 3, 4, 5, and 6 [always bothered]”) for each of ten questions. The total score is the sum of the numeric value of each answered question. The domain scores are sums of the following: disease domain (questions 1 to 3), mood/emotional distress domain (questions 4 to 6), and social functioning domain (questions 7 to 10). The Skindex-10 has been found to correlate with both itch intensity as well as other instruments evaluating quality of life (Ref.4).

### Treatment Satisfaction

On Day 15 or at the time of early termination, patients were asked to provide an evaluation of the study drug by answering the following question: “How would you rate the study medication?”—4 Excellent; 3 Very Good; 2 Good; 1 Fair or 0 Poor.

### Safety Assessments

The safety assessments taken for each patient were the following:
Incidence and severity of AEs and SAEs
Physical examination;
Vital signs;
Oxygen saturation (Part A only);
12-lead ECG;
Clinical laboratory tests; and
Urine volume (Part A only).

### Physical Examination

Physical examinations included, at minimum, an examination of the heart, lungs, abdomen, extremities, neurological system, and vascular system. Clinically significant abnormalities prior to administration of the first dose of study drug were reported as medical history and clinically significant new or worsening findings observed after the first dose of study drug are reported as adverse events (AEs).

### Vital Signs

Vital signs included body temperature, HR, sitting or semi-recumbent BP, and RR. In Part A, oxygen saturation was also measured via pulse oximetry.

Measurements were repeated if a value was out of the reference range and additional measurements were taken at other times if judged to be clinically appropriate.

Measurement were taken at nominal time (±5 min).

### Electrocardiogram

Twelve-lead ECGs were obtained and read locally by the Investigator or physician designee. Clinically significant abnormalities prior to administration of the first dose of study drug were reported as medical history and clinically significant new or worsening findings observed after the first dose of study drug were reported as AEs.

### Clinical Laboratory Tests

The following clinical laboratory tests were performed and analyzed by one of the central laboratories. Processing and shipment of central laboratory samples was according to the Laboratory Manual protocols.
Hematology: hemoglobin, hematocrit, platelet count, white blood cell (WBC) count (including differential);
Serum Chemistry: total bilirubin, direct bilirubin (if total bilirubin is outside the ULN) and alkaline phosphatase alanine transaminase (ALT), aspartate aminotransferase (AST), glucose (non-fasting), serum creatinine, blood urea nitrogen (BUN), electrolytes (sodium, potassium, chloride, calcium and phosphorus), parathyroid hormone (PTH; Part B, on Day 1 only).
Additionally, serum sodium was measured in Part A
Urine Chemistry: Urine sodium and creatinine (Part A only, as applicable)
Serum Pregnancy: In women of childbearing potential
Follicle Stimulating Hormone: In women who had been amenorrheic for at least 1 year and were between 45 and 55 years of age to confirm that they were not of childbearing potential
Blood Hepcidin: Was analyzed in serum in Part A only.

### 24-Hour Urine Sample (Part A Only)

Patients were instructed to collect all urine over a 24-hour period for urine sodium, creatinine, and volume while in the clinical research unit. The collection started on the day prior to dialysis and ended prior to the start of dialysis the next day. Fluid intake was also measured during this period.

### Adverse Events (AEs)

### DEFINITION OF ADVERSE EVENTS

A treatment-emergent AE (TEAE) is defined as any untoward medical occurrence in a patient administered a pharmaceutical product, and does not necessarily have a causal relationship with the treatment. An AE can be any unfavorable and unintended sign (e.g., including an abnormal laboratory finding), symptom, or disease temporally associated with the use of the study drug, whether or not it is considered to be study drug related.

This definition of a TEAE includes any newly occurring event or previous condition that has increased in severity or frequency since the administration of study drug.

Abnormal laboratory tests at Screening or before the administration of the study drug that were assessed as clinically significant were not reportable as AEs. Clinically significant abnormalities prior to administration of the first dose of study drug were reported as medical history, unless they were expected findings from medical history that had already been reported (e.g., elevated PTH in a patient with a medical history of hyperparathyroidism).

AEs resulting from concurrent illnesses, reactions to concurrent illnesses, reactions to concurrent medications, or progression of disease states were also be recorded. In order to avoid vague, ambiguous, or colloquial expressions, AEs were recorded in standard medical terminology rather than the patient's own words. Signs and symptoms were reported individually unless, in the judgment of the Investigator, they could be grouped under a widely accepted inclusive term (e.g., gastroenteritis in lieu of abdominal pain, nausea, vomiting, and diarrhea).

An abnormal result of a diagnostic procedure following administration of the study drug is captured as an AE if the finding meets the following criteria:
Results in study withdrawal;
Is associated with signs or symptoms;
Is considered by the Investigator to be of clinical significance.

The underlying diagnosis as captured as the AE, not the procedure itself.

Overdose was defined as an accidental or intentional exposure to study drug at a dose higher than specified in the protocol or higher than known therapeutic dose. Any overdose of study drug was reportable as a protocol deviation. Any overdose associated with clinical signs or symptoms, were captured as AEs.

### Pharmacokinetic Evaluation (Part A Only)

For PK analysis, approximately 4 mL of blood was collected from the predialyzer (arterial) line at the following times:
Day 1: Within 5 min. prior to starting dialysis; and within 10 min. following the end of dialysis (i.e. from return of blood to patent).
   At 5 mins., 15 mins. and 30 mins. and at 1, 2, 3, 4, 6, 8 12 and 24 hours after injection of CR845; (i.e. t=0). A ±1 minute window is allowed for PK blood draws up to 30 min (inclusive), a ±5 minute window was allowed for the remainder of the PK assessments.
Day 3: Within 5 min. prior to starting dialysis;
Day 5: Within 5 min. prior to starting dialysis; and within 10 min. following the end of dialysis (i.e. from return of blood to patent).
   At 5 mins., 15 mins. and 30 mins. and at 1, 2, 3, 4, 6, 8 12 and 24 hours after injection of CR845; (i.e. t=0). A ±1 minute window was allowed for PK blood draws up to 30 min (inclusive), a ±5 minute window was allowed for the remainder of the PK assessments.
Day 8: Within 5 min. prior to starting dialysis; and within 10 min. following the end of dialysis (i.e. from return of blood to patent).

Plasma samples were analyzed for CR845 using liquid chromatography with tandem mass spectrometric detection (LC/MS/MS) according to validated analytical methods. Blood samples were collected in 4 mL lavender-top vacutainer tubes containing K2EDTA as the anticoagulant. The total blood volume collected for CR845 PK sampling was approximately 108 mL. Blood samples were placed on ice immediately after collection and remain on ice throughout processing. Blood samples were then centrifuged according to standard phlebotomy sample collection procedures. The study drug is stable in whole blood on ice for at least 1 hour, so sample processing was completed no later than 1 hour from the collection time. The resulting plasma was transferred in aliquots of approximately 1 mL each into two appropriately labeled polypropylene screw-cap tubes. All sample collection and freezing tubes were to be clearly labeled. Plasma samples are frozen at −70° C. or below. The samples were frozen within 1 hour of collection and remain frozen until assayed. The actual time of collection was recorded. Samples were shipped frozen on dry ice to an analytical laboratory.

### Statistical Methods

### General Considerations

All statistical tests are performed at the α=0.05 significance level using one-sided tests, unless otherwise noted.

### Interim Analysis

An unblinded interim analysis was performed following the completion of Part A. There was no interim analysis for the Part B of the study.

### Analysis Populations

Four analysis populations were defined as follows:
Safety Population (Parts A and B): All patients who were randomized and had received any study drug at any time during the study comprise the safety population. Within the safety population, treatment assignment was made on an “as treated” basis.
Pharmacokinetic Evaluable Population (Part A only): The PK evaluable population included all patients who received CR845 and have sufficient plasma concentrations for PK analysis are included in the PK population.
Modified Intent-To-Treat (MITT) Population (Part B only): All patients who were randomized and received at least one dose of the study drug, and at least one post-baseline efficacy assessment constituted the MITT population.

Assignment of patients to treatment condition was made on an “as randomized” basis.
Per Protocol (PP) Population (Part B only): All patients who met the MITT population criteria without major protocol deviations adversely affecting the efficacy data analysis constituted the PP population.

The MITT population was used for the summaries of protocol deviations, demographics, and baseline disease characteristics. The safety population was used for the summaries of all safety assessments. The MITT and PP populations were used for the analysis and summaries of efficacy endpoints. The PK evaluable population was used for all PK data summary.

Data from placebo-treated patients from the different dosing groups in Part A were combined for analysis and reporting.

### Patient Disposition

The number of patients randomized, completed, or discontinued from the study, along with the reason for discontinuation, was presented overall and by treatment group. Patient count by analysis population was also tabulated.

### Efficacy Analysis

### Primary Efficacy Endpoints (Part B)

The primary efficacy endpoint was the change from baseline to the average of the Week 2 worst itching VAS. The baseline for the VAS score is defined as the average of all respective assessments during the Run-In period. The mean of the last 8 VAS scores from Week 2 was used for the comparison.

Mixed model for repeated measures (MMRM) analysis was applied with the daily worst itching VAS scores during the 2-week treatment period serving as the dependent variable. The model included the baseline VAS score as covariate, treatment, week (Weeks 1 and 2), day within week (Days 1 through 7)), the treatment by week interaction as fixed effects, and patient identification as the unit for repeated measures. The between treatment difference was estimated as the simple contrast at Week 2 in the model treatment parameter. The primary analysis was based on the MITT population and the sensitivity analysis was based on the per protocol population.

To explore the robustness of the results, other statistical method, such as analysis of covariance (ANCOVA) or missing data imputation was also applied to this endpoint.

### Other Efficacy Endpoints and Exploratory Endpoints (Parts A and B)

Endpoints of a continuous data nature were analyzed similarly as for the primary endpoint. Endpoints of ordinal or categorical data nature are analyzed using generalized linear model (e.g., logistical regression) or non-parametric test.

### Pharmacokinetic Analysis (Part A)

Raw serum values and PK parameters for CR845 were summarized using appropriate descriptive statistics. Plasma concentrations were summarized descriptively and graphically by nominal time. Pharmacokinetic parameters (half-life, Cmax, Tmax, AUC, Vd, etc) were calculated from the plasma concentration data using validated software such WinNonlin. Individual plasma CR845 concentrations were listed and plotted by patient. Statistical tests were applied to raw values and PK parameters of each sample to obtain a full PK profile analysis.

### Ethics

The study was conducted in accordance with ethical principles founded in the Declaration of Helsinki (Edinburgh 2000) and all accepted amendments, the ICH principles of GCP (including archiving of essential study documents), and the applicable regulations of the country in which the study is conducted. The protocol, the Investigator's informed consent document, and related patient information and recruitment materials were reviewed and approved by an Institutional Review Board (IRB) or Independent Ethics Committee (IEC), before the start of the study.

### Good Clinical Practices (GCP)

The study was conducted in accordance with the ICH for GCP and the appropriate regulatory requirement(s).

### Institutional Review Board (IRB)

The IRB reviewed and approved the protocol, ICF, and related patient information and recruitment materials before the start of the study.

### Results of Clinical Trial

The above-described randomized, double-blind, placebo controlled study was run with sixty-five patients receiving a dose of either CR845 or placebo after each dialysis session, three times per week. The trial was run in twenty one clinical centers in the United States. Thirty-three of the sixty-five patients in the trial received CR845, the remaining thirty-two patients received placebo. Three patients terminated early and did not complete the study.

Fifty-seven patients, thirty receiving CR845 and twenty-seven receiving placebo, complied with the protocol. Fifty-six patients received all six planned doses of CR845 (twenty-six patients) or placebo (thirty patients). One patient received five of the six doses of placebo and two patients received five of the six doses of CR845. One patient received four doses and two patients received three of the six planned doses of placebo. One patient received two of the six planned doses of placebo, and one patient received only one of the six planned doses of CR845.

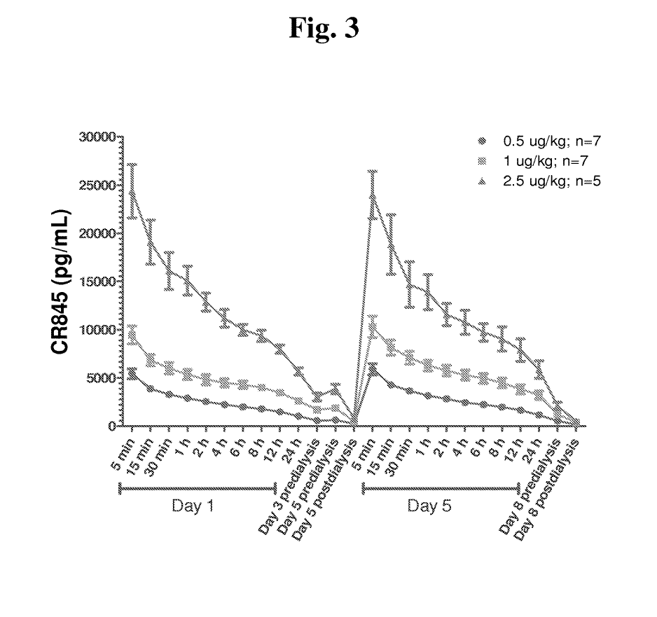

FIG. 3 shows the pharmacokinetic profile for each of the 0.5 mg/kg, 1.0 mg/kg and 2.5 mg/kg doses of CR845 over days two and five of the trial and tracks the level of CR845 in patient blood before and after dialysis. Surprisingly, the area under the curve (AUC), an indicator of bioavailability, is ten-fold greater in these dialysis patients than in health volunteers. This suggests that loss of kidney function extends the half-life of CR845 in the blood of dialysis patients and makes patients suffering from chronic kidney dysfunction well suited for CR845 treatment.

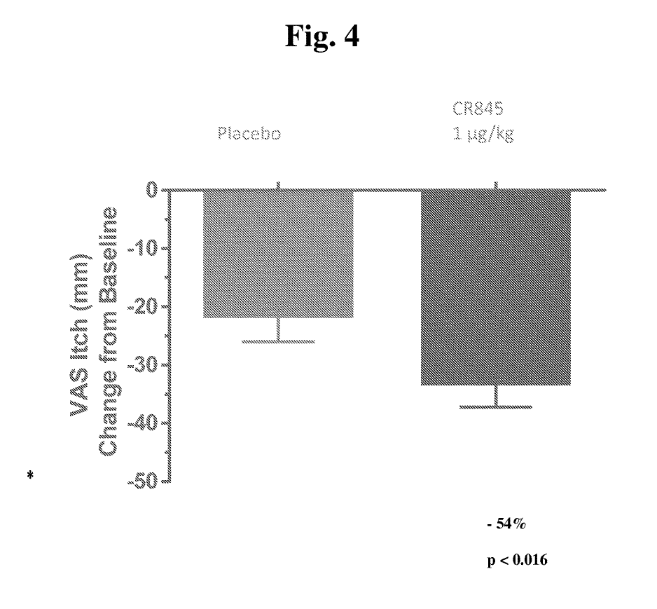

The change in itch from baseline determined pre-trial on a visual analog scale (VAS) as indicated by placebo-treated dialysis patients and dialysis patients treated with 1 ug/kg CR845 is shown in FIG. 4. CR845 treatment resulted in a 54% reduction in itch, which was highly significant at the p=0.016 level.

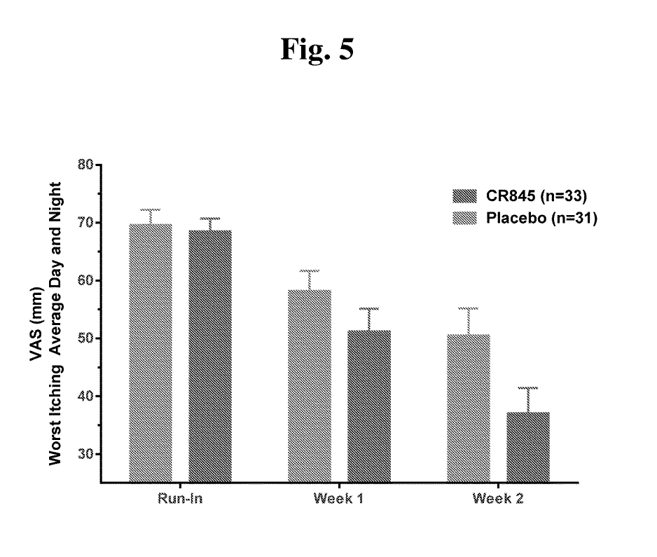

FIG. 5 shows the change in itch in placebo-treated and CR845-treated dialysis patients with end stage renal disease (ESRD) during week prior to clinical trial (the so-called run-in period), and at week 1 and week 2 of the CR845-treatment trial. The level of itch determined on a visual analog scale (VAS) in CR845-treated patients was 62% lower than in placebo-treated patients, significant at the p<0.05 level.

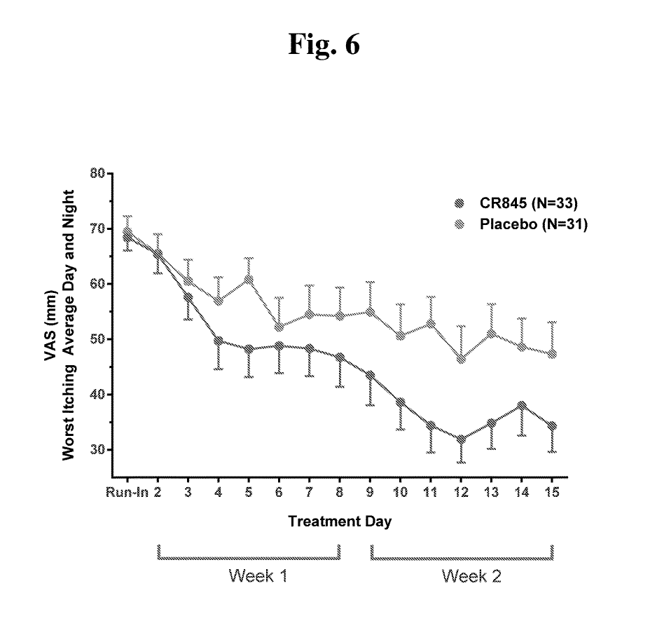

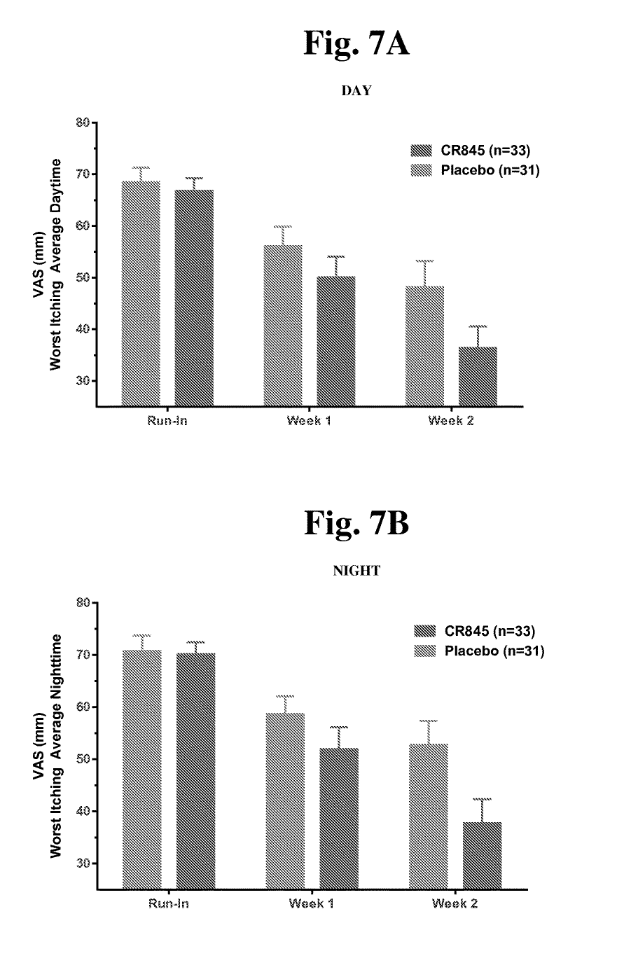

The average of “worst itching” (i.e. the highest patient-scored itch) over daytime and night time as assessed by a visual analog scale (VAS) in CR845-treated dialysis patients was monitored over the 15 days of the trial (see FIG. 6). The reduction of itch intensity in dialysis patients treated with CR845 continued throughout the two week treatment period. Dialysis patients who received placebo experienced an initial improvement in itching in the first week, which reached a plateau in the second week as might be expected for a placebo effect. Worst daytime and night time itch levels are shown in FIGS. 7A and 7B, respectively, for CR845-treated and placebo treated dialysis patients over the three week period from the run-in week prior to initiation of the trial, throughout the two weeks of the trial. The average itch reduction over both daytime and night time was found to be 48% from baseline. Significantly, the observed 75% reduction in worst night time itch was highly significant (p=0.007), and daytime reduction of 51% was also significant (p=0.03).

Quality of life effects due to treatment with CR845 was assessed by the Skindex-10 questionnaire (see above) divided into three domains: The first domain was the “Disease Domain” consisting of three questions; the “Mood/Emotional Distress” domain also consisting of three questions; and the “Social Functioning Domain” consisting of four questions.

Each of the ten questions ask “during the past WEEK, how often have you been bothered by” the following questions on a scale of 0=Never bothered to 6=always bothered:

I. “Disease Domain”
1. . . . Your itching.
2. The persistence/recurrence of your itching.
3. The appearance of your skin from scratching.

II “Mood/Emotional Distress”
4. Frustration about your itching.
5. Being annoyed about your itching.
6. Feeling depressed about your itching.

III. “Social Functioning”
7. Feeling embarrassed about your itching.
8. The effects of your itching on your interactions with others.
9. The effects of your itching on your desire to be with people.
10. The effects of your itching making it hard to work or do what you enjoy.

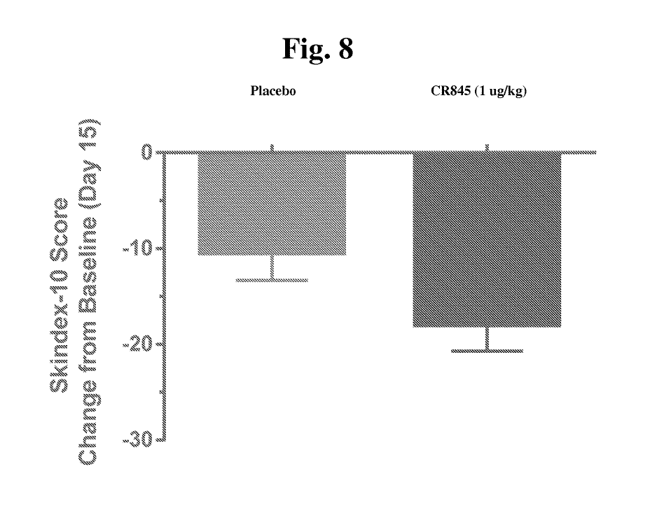

The overall change in Skindex scores from baseline determined at day 1 for the placebo-treated patients and the patients treated with 1 ug/kg CR845 from day 1 through day 15 of the trial is shown in FIG. 8. Treatment with 1 ug/kg CR845 resulted in a 71% reduction in Skindex-10 scores compared with placebo with a significance of p=0.031.

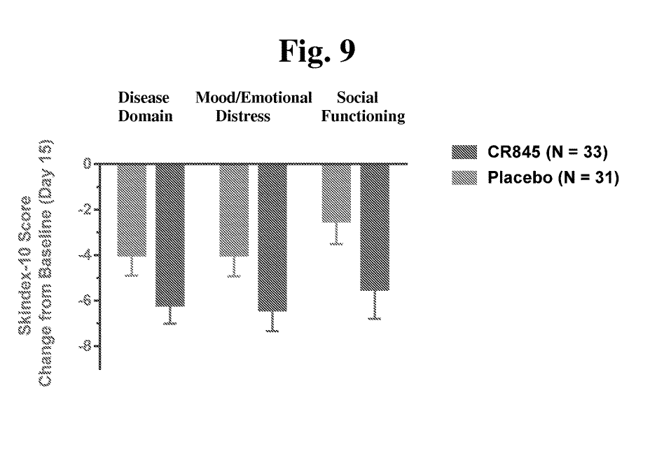

Skindex-10 scores for each if the three domains: Disease; Mood/Emotional Distress; and Social Functioning; for CR845-treated and placebo-treated dialysis patients are shown in FIG. 9. In each domain, CR845-treatment resulted in a trend toward lower Skindex-10 scores (i.e. a surrogate measure for improved quality of life) in all aspects represented by the three tested domains. Mood/emotional distress reduction was significant (p=0.046).

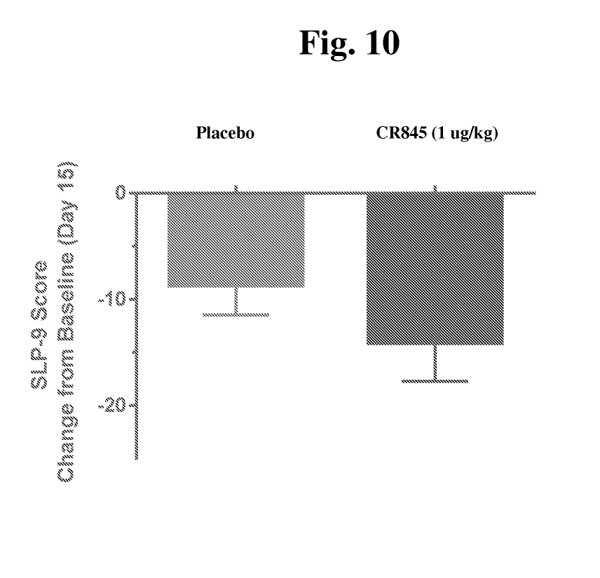

The itch MOS sleep index (SLP-9: See above) was used to track trends in the effect of CR845 treatment on itch-related sleep disturbance. FIG. 10 shows the compiled results of the SLP-9 score over the 15 day treatment period of the trial for placebo-treated and CR845-treated dialysis patients. The results show a 62% reduction (i.e. improvement) in sleep as represented by SLP-9 score associated with CR845-treatment as compared with placebo.

In this clinical trial, I.V. CR845 was shown to be safe and well tolerated with no CR845-related serious adverse events (AEs) reported. The most common AEs were transient numbness and dizziness, with no episodes of CNS side effects (e.g., dysphoria and hallucinations).

The disclosures of each of the patents and non-patent references cited in this application are incorporated by reference herein in their entireties.

### REFERENCES

1. Investigator's Brochure for CR845. Edition No. 6, June 2014, Cara Therapeutics, Inc.
2. Wikström B, Gellert R, Ladefoged S D, Danda Y, Akai M, Ide K, Ogasawara M, Kawashima Y, Ueno K, Mori A, Ueno Y. Kappa-opioid system in uremic pruritus: multicenter, randomized, double-blind, placebo-controlled clinical studies. J. Am. Soc. Nephrol. 2005; 16(12):3742-47.
3. Mathur V S, Lindberg J, Germain M, Block G, Tumlin J, Smith M, Grewal M, McGuire D. A longitudinal study of uremic pruritus in haemodialysis patients. Clin. J. Am. Soc. Nephrol. 2010; 5:1410-1419.
4. Kumagai H, Ebata T, Takamori K, Muramatsu, T, Nakamoto H, Suzuki H. Effect of a novel kappa-receptor agonist, nalfurafine hydrochloride, on severe itch in 337 haemodialysis patients: a Phase III, randomized, double-blind, placebo-controlled study. NDT. 2009; April; 25(4): 1251-1257.
5. Ständer S, Richter L, Osada N, Metze D. Hydroxyethyl Starch-induced Pruritus: Clinical Characteristics and Influence of Dose, Molecular Weight and Substitution. Acta Derm Venereol. 2013 Sep. 16.
6. Pisoni R L, Wikstrom B, Elder S J, Akizawa T, Asano Y, Keen M L, Mendelssohn D C, Young E W, and Port F K. Pruritus in haemodialysis patients: international results from the Dialysis Outcomes and Practice Patterns Study (DOPPS). NDT. 2006; 21: 3495-3505.

1. A method of prevention, inhibition or treatment of uremic pruritus in a dialysis patient, the method comprising administering an effective amount of a kappa opioid receptor agonist to a patient undergoing a dialysis regimen on at least one of the days in which the dialysis occurs.

2. The method of claim 1, wherein the kappa opioid receptor agonist is administered on at least two days in which the dialysis occurs.

3. The method of claim 1, wherein the kappa opioid receptor agonist is administered three times per week for at least one week.

4. The method of claim 1, wherein at least one administration of the kappa opioid receptor agonist is within one hour after the dialysis.

5. The method of claim 1, wherein at least one administration of the kappa opioid receptor agonist is by intravenous injection.

6. The method of claim 5, wherein the intravenous injection is by an I.V. bolus injection.

7. The method of claim 1, wherein the effective amount of the kappa opioid receptor agonist is administered estimated from a patient's dry weight.

8. The method of claim 7, wherein the effective amount of the kappa opioid receptor agonist administered is from about 0.1 μg/kg of patient's dry weight to about 5.0 μg/kg of the patient's dry weight.

9. The method of claim 8, wherein the effective amount of the kappa opioid receptor agonist administered is from about 0.5 μg/kg of patient's dry weight to about 2.5 μg/kg of the patient's dry weight.

10. The method of claim 9, wherein the effective amount of the kappa opioid receptor agonist administered is about 2.5 μg/kg of patient's dry weight.

11. A method of inhibition or treatment of an adverse symptom associated with dialysis in a patient undergoing a dialysis, the method comprising administering an effective amount of a kappa opioid receptor agonist to the patient undergoing the dialysis.

12. The method according to claim 11, wherein the adverse symptom is selected from the group consisting of uremic pruritus, sleep disruption, and mood alteration.

13. The method according to claim 12, wherein the adverse symptom is sleep disruption.

14. The method according to claim 13, wherein the adverse symptom is pruritus-associated sleep disruption.

15. The method according to claim 12, wherein the mood alteration is depression.
